(19) United States
(12) Patent Application Publication
(10) Pub. No.: US 2008/0221660 A1
(43) Pub. Date: Sep. 11, 2008

(54) Platelet Gel for Treatment of Aneurysms

(75) Inventors: Jack Chu, Santa Rosa, CA (US); Jonathan Morris, Santa Rosa, CA (US); Josiah Wilcox, Santa Rosa, CA (US); Brian Fernandes, Roseville, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/045,909

(22) Filed: Mar. 11, 2008

Related U.S. Application Data
(63) Continuation-in-part of application No. 10/977,545, filed on Oct. 28, 2004.

(51) Int. Cl. A61F 2/82

(52) U.S. Cl. 623/1.13; 623/1.42; 623/1.41

(57) ABSTRACT

Methods for ameliorating stent graft migration and endoleak using treatment site-specific platelet gel compositions in combination with stent grafts are disclosed. Also disclosed are platelet gel compositions directly to treatment sites before, during or after stent graft implantation. Additional embodiments include medical devices having platelet gel coatings and/or platelet gel delivery devices useful for treating aneurysms.

### CROSS-REFERENCE TO RELATED APPLICATIONS

The present application is a continuation-in-part of U.S. patent application Ser. No. 10/977,545 filed Oct. 28, 2004 which is incorporated by referenced herein in its entirety.

### FIELD OF THE INVENTION

Methods for preventing stent graft migration and endoleak by promoting tissue in-growth on the stent graft are provided. Specifically, methods for applying or forming platelet gel directly on stent grafts or directly to treatment sites before, during or after stent graft implantation are provided. More specifically, medical devices having platelet gel coatings and/or platelet gel delivery devices useful for treating aneurysms are provided. Optionally, the platelet gel coatings further contain one or more bioactive agents.

### BACKGROUND

The threshold size for treating aneurysms arises when a thinning, weakening section of an artery wall balloons out to more than 150% of the artery's normal diameter. The most common and deadly of these occur in the aorta, the large blood vessel stretching from the heart to the lower abdomen. A normal aorta is between 1.6 to 2.8 centimeters wide; if an area reaches as wide as 5.5 centimeters, the risk of rupture increases such that surgical treatment is recommended. Aneurysms are asymptomatic and often burst before the patient reaches the hospital.

Aneurysms are estimated to cause approximately 32,000 deaths each year in the United States. Additionally, aneurysm deaths are suspected of being underreported because sudden unexplained deaths, about 450,000 in the United States alone, are often simply misdiagnosed as heart attacks or strokes while many of them may be due to aneurysms. Aneurysms most often occur in the aorta, the largest artery in the body. Most aortic aneurysms, approximately 15,000/year, involve the abdominal aorta while approximately 2,500 occur in the chest. Cerebral aneurysms occur in the brain and present a more complicated case because they are more difficult to detect and treat, causing approximately 14,000 U.S. deaths per year. Aortic aneurysms are detected by standard ultrasound, computerized tomography (CT) and magnetic resonance imaging (MRI) scans and the increased use of these scanning techniques for other diseases has produced an estimated 200% increase in the diagnosis of intact aortic aneurysms. Approximately 200,000 intact aortic aneurysms are diagnosed each year due to this increased screening alone.

U.S. surgeons treat approximately 50,000 abdominal aortic aneurysms each year, typically replacing the abnormal section with a plastic or fabric graft in an open surgical procedure. A less-invasive procedure that has recently become more popular uses a stent graft which while compressed in a tubular catheter is threaded through the arteries to the aneurysm and is deployed to span the aneurysm to provide aortic support without open surgery. A vascular graft containing a stent (stent graft) is placed within the artery at the site of the aneurysm and acts as a barrier between the blood and the weakened wall of the artery, thereby decreasing pressure on artery. The less invasive approach of stent grafting aneurysms decreases the morbidity seen with conventional open surgical aneurysm repair. Additionally, patients whose multiple medical comorbidities make them excessively high risk for conventional aneurysm repair are candidates for stent grafting. Stent grafts have also emerged as a new treatment for a related condition, acute blunt aortic injury, where trauma causes damage to the artery. There are, however, risks associated with endovascular repair of abdominal aortic aneurysms. A common risk is migration of the stent graft due to hemodynamic forces within the artery. Graft migrations lead to endoleaks, a leaking of blood into the aneurysm sac between the outer surface of the graft and the inner lumen of the blood vessel.

The abdominal aorta between the renal artery and the iliac branch is the most susceptible arterial site to aneurysms. While this area of the aorta is ideally straight, in many patients the aorta is curved leading to asymmetrical hemodynamic forces. When a stent graft is deployed in this curved portion of the aorta, hemodynamic forces are uneven on the graft which can, lead to graft migration. Additionally, the asymmetrical hemodynamic forces can cause remodeling of the aneurysm sac which can lead to increased risk of aneurysm rupture and increased endoleaks.

One goal of endovascular repair of aorta aneurysms is to provide a graft positioned in close contact with the vessel wall, and is in fact, sealed to the vessel wall. The greater the area of the stent graft in contact with the artery wall, the better graft fixation, and tighter the seal which leads to a decreased risk of migration and endoleak. Endoleaks present a risk factor for post-surgical rupture of the aneurysm due to increased blood pressure within the aneurysm sac.

Existing stent grafts have been designed with stainless steel anchoring barbs that engage the aortic wall directly to prevent migration. Additionally, endostaples have been developed to fix the graft more readily to the treatment site. These physical anchoring techniques have proven to be effective in some patients; however, they have not sufficiently ameliorated all the stent graft migration and endoleak problems associated with current stent-grafting methods and devices.

The combination of the magnetizable metal scaffolding of most stent grafts and a predilection to graft migration has led to the contraindication of magnetic resonance imaging (MRI) in some patients having stent grafts. The magnetic fields used in this imaging process, when moving across the body, may cause insufficiently endothelialized magnetizable metal-containing stents to migrate. Anchoring the stent graft into the vessel wall may be expected to ameliorate this problem to the extent that sufficient tissue in-growth occurs. Inducing significant endothelialization of the stent graft may reduce the risk of migration and allow patients access to this vital medical diagnostic procedure.

Therefore there exists a medical need for compositions useful for coating stent grafts or direct application to the aneurysm wall at the time of stent graft implantation that promote healing, reduce endoleaks and minimize device migration by promoting endothelial tissue in-growth.

### SUMMARY OF THE INVENTION

Compositions are provided in combination with vascular stent grafts for the treatment of aneurysms. Additionally, devices are described which provide structural support for weakened arterial walls while the accompanying compositions seal the support to the tissue wall and promote tissue in-growth to reduce graft migration and prevent endoleaks. In further embodiments, the platelet gel can comprise one or more bioactive agents.

In one embodiment, a stent graft is described comprising: an abluminal surface; a luminal surface; and platelet gel on at least one of said abluminal and said luminal surfaces, wherein said platelet gel further comprises a bioactive agent and wherein said abluminal surface of said stent graft is coated with platelet gel prior to deployment by depositing platelet plasma and thrombin on said stent graft compressed within the stent graft chamber of a stent deployment catheter. In one embodiment, the platelet gel is applied directly to said stent graft compressed within a stent deployment catheter.

In one embodiment, the platelet gel comprises thrombin and platelet plasma. In another embodiment, the platelet plasma comprises at least one of platelet rich plasma or platelet poor plasma. In one embodiment, the platelet plasma and/or the thrombin are autologous.

In one embodiment, the platelet gel further comprises one or more bioactive agents selected from the group consisting of small molecules, peptides, proteins, hormones, DNA or RNA fragments, cells, genetically engineered cells, genes, cell growth promoting compositions and matrix metalloproteinase inhibitors.

Described herein is a method for providing a stent graft and platelet gel to a treatment site comprising: delivering a stent graft to an aneurysm site; and delivering to the abluminal surface of said stent graft thrombin and platelet plasma such that platelet gel is formed between said abluminal surface of said stent graft and the blood vessel wall. In one embodiment, the platelet gel substantially fills said aneurysm sac. In another embodiment, the platelet plasma comprises at least one of platelet rich plasma and platelet poor plasma. In one embodiment, the thrombin and/or said platelet plasma are autologous.

In one embodiment, the platelet gel further comprises one or more bioactive agents is selected from the group consisting of small molecules, peptides, proteins, hormones, DNA or RNA fragments, cells, genetically engineered cells, genes, cell growth promoting compositions and matrix metalloproteinase inhibitors.

In one embodiment, the method further comprises: advancing a stent deploying catheter containing a stent graft to a treatment site; advancing at least one injection catheter containing at least one component of platelet gel to said treatment site; deploying said stent graft at said treatment site; and applying said components of said platelet gel from said at least one injection catheter to said inner lumen of said blood vessel at said treatment site to form platelet gel; such that said abluminal surface of said stent graft engages said platelet gel and said blood vessel luminal surface contacts said platelet gel at said treatment site. In one embodiment, the step of applying said components includes applying cell selected from among a list consisting of: stem cells, adipose stem cells, mesenchymal stem cells, and cells from bone marrow.

In one embodiment, the injection catheter is selected from the group comprising single lumen injection catheter and multilumen injection catheter. In another embodiment, a first of the at least one injection catheter reaches the treatment site through a different route than a second of the at least one injection catheter. In another embodiment, the first of the at least one injection catheter reaches the treatment site through a blood vessel bisecting the treatment site thereby delivering the cell growth promoting composition directly to the aneurysm sac.

In one embodiment, the treatment site is selected from the group consisting of the area where the proximal end of the stent graft contacts the vessel lumen wall, the junction between a stent graft and an iliac limb section, the aneurysm sac, and combinations thereof.

In one embodiment, the thrombin is of bovine origin. In another embodiment, the thrombin is of recombinant human origin.

A method is described herein for providing a stent graft and platelet gel to aneurysm site comprising: loading a stent graft into a delivery catheter, said stent graft comprising an abluminal surface and a luminal surface; applying to at least one of said abluminal surface and said luminal surface, thrombin and platelet plasma to form platelet gel on said stent graft within said delivery catheter; advancing said deployment catheter to said aneurysm site; and deploying said stent graft at said aneurysm site. In another embodiment, the platelet plasma comprises at least one of platelet rich plasma or platelet poor plasma. In another embodiment, the platelet plasma and or said thrombin are autologous. In another embodiment, the platelet gel further comprises one or more bioactive agents selected from the group consisting of small molecules, peptides, proteins, hormones, DNA or RNA fragments, cells, genetically engineered cells, genes, cell growth promoting compositions and matrix metalloproteinase inhibitors.

### BRIEF DESCRIPTION OF THE FIGURES

FIG. 1 depicts a fully deployed stent graft with an exterior metal scaffolding as used in an abdominal aortic aneurysm.

FIG. 2 depicts a stent graft delivery catheter adapted to allow coating of the stent graft with platelet gel on the abluminal surface within the delivery catheter.

FIG. 3 depicts an alternative stent graft delivery catheter adapted to allow coating of the stent graft with platelet gel on the abluminal surface within the delivery catheter.

FIG. 4a-c depict an alternative stent graft delivery catheter adapted to allow coating of the stent graft with platelet gel on the abluminal surface within the delivery catheter.

FIG. 5a-b depict a stent graft delivery catheter adapted to allow coating of the stent graft with platelet gel on the luminal surface within the delivery catheter.

FIG. 6a-c depict deployment of a stent graft and an injection catheter suitable for delivery of platelet gel to a treatment site.

FIG. 7a-b depict a method of delivering platelet gel directly into the aneurysm sac after deployment of a stent graft.

FIG. 8a-c depict an alternate method of delivering platelet gel directly into the aneurysm sac after deployment of a stent graft.

FIG. 9 depicts an alternate method of delivering platelet gel directly into the aneurysm sac after deployment of a stent graft.

FIG. 10 depicts an alternate method of delivering platelet gel directly into the aneurysm sac after deployment of a stent graft.

FIG. 11 depicts an alternate method of delivering platelet gel directly into the aneurysm sac after deployment of a stent graft.

FIG. 12 depicts the effects of the autologous platelet gel on arterial smooth muscle cell proliferation.

FIG. 13 depicts the effects of the autologous platelet gel on endothelial cell proliferation.

FIG. 14 depicts the effects of the autologous platelet gel on fibroblast cell proliferation.

FIG. 15 depicts the effects of platelet poor plasma on human dermal fibroblast growth.

FIG. 16 depicts the effects of the autologous platelet gel on endothelial cell migration.

FIG. 17a-b depict the tissue response to implantation of the autologous platelet gel (FIG. 17a) or Matrigel® (FIG. 17b) in athymic mice.

FIG. 18a-b depict the quantity of protein released over time from platelet poor plasma (PPP) gels (18a) and platelet rich plasma (PRP) gels (18b), containing various of dexamethasone (mg).

FIG. 19a-d depict the cumulative release of dexamethasone phosphate (DexP) and dexamethasone acetate (DexAc) over time at various Dex loads in platelet poor plasma (PPP) gels (19a and 19c) and platelet rich plasma (PRP) gels (19b and 19d).

FIG. 20a-b depict DexP (20a) and DexAc (20b) stability in dilute and concentrated PPP as well as phosphate buffered saline (PBS).

FIG. 21 depicts cell proliferation after 48 hours for various mixtures of DexP, DexAc, doxycline monohydrate (DoxHy), growth media (GM), and PRP.

FIG. 22 depicts the doxycline hydrochloride (DoxHCl) release from 5 mg and 10 mg microspheres (MSP) loaded into PPP gels.

### DEFINITION OF TERMS

Prior to setting forth embodiments, it may be helpful to an understanding thereof to set forth definitions of certain terms that will be used hereinafter:

Animal: As used herein “animal” shall include mammals, fish, reptiles and birds. Mammals include, but are not limited to, primates, including humans, dogs, cats, goats, sheep, rabbits, pigs, horses and cows.

Bioactive Agents(s): As used herein “bioactive agent” shall include any compound or drug having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, matrix metalloproteinase inhibitors and transforming nucleic acids. Bioactive agents can also include anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like. Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Endoleak: As used herein “endoleak” refers to Type I endoleaks, i.e., the presence of flow of blood past the seal between the proximal end of the stent graft and the vessel wall, and into the aneurysmal sac, when all such flow should be contained within its lumen.

Migration: As used herein “migration” refers to displacement of the stent graft sufficient to be associated with another complication, for example, an endoleak.

Treatment Site: As used herein “treatment site” shall mean an aneurysm site, acute traumatic aortic injury or other vascular-associated pathology. Treatment site can also refer to a delivery site for platelet gel including, but not limited to, an aneurysm sac, the proximal end of a deployed stent graft, the distal end of a deployed stent graft, areas of overlap by two stent graft portions, and portions of a deployed stent graft adjacent to a blood vessel wall.

### DETAILED DESCRIPTION

Some embodiments provide compositions, devices and related methods useful for preventing implantable medical device post-implantation migration and endoleak. More specifically, the compositions, devices and related methods promote implantable medical device attachment to blood vessel luminal walls. In certain embodiments, methods and compositions useful for minimizing post-implantation stent graft migration following deployment at an aneurysmal treatment site and are also useful in preventing or minimizing post-implantation endoleak following stent-graft deployment at an aneurysmal treatment site are provided.

For convenience, the devices, compositions and related methods discussed hereinafter will be exemplified using stent grafts intended to treat aneurysms. As discussed briefly above, an aneurysm is a swelling, or an expansion of the blood vessel lumen at a defined point and is generally associated with a vessel wall defect. Aneurysms are often a multi-factorial asymptomatic vessel condition that if left unchecked may result in spontaneous rupture, often with fatal consequences. Previous methods to treat aneurysms involved highly invasive open surgical procedures where the affected vessel region is opened and removed and/or replaced with a synthetic graft that is sutured in place. However, this procedure is extremely risky and is generally only employed in otherwise healthy vigorous patients who are expected to survive the associated surgical trauma. Elderly and more feeble patients were not candidates for open aneurysmal surgeries and remained untreated and thus at continued risk for sudden death.

To overcome the risks associated with invasive open aneurysmal surgeries, stent grafts were developed that can be deployed with a cut down procedure or percutaneously using minimally invasive procedures. Essentially, a catheter having a stent graft compressed and fitted into the catheter's distal tip is advanced through an artery to the aneurysmal site. The stent graft is then deployed within the vessel lumen juxtaposed to the weakened vessel wall forming an inner liner that insulates the aneurysm from the body's hemodynamic forces thereby reducing, or eliminating, the possibility of rupture. The size and shape of the stent graft is matched to the treatment site's lumen diameter and aneurysm length. Moreover, bifurcated grafts are commonly used to treat abdominal aortic aneurysms that are located near and must span the arteries at the iliac branch.

Stent grafts generally comprise a metal scaffolding supporting biocompatible graft material such a Dacron® or a fabric-like material woven from a variety of biocompatible polymer fibers. Other embodiments include extruded sheaths and coverings. The scaffolding is often positioned on the luminal wall-contacting surface of the stent graft and directly contacts the vessel lumen. The sheath material is stitched, glued or molded onto the scaffold. In other embodiments, the scaffolding may be on the graft's blood flow contacting surface or interior. When a self-expanding stent graft is deployed from the delivery catheter, the scaffolding expands to fill the lumen and exerts circumferential force against the lumen wall. This circumferential force is generally sufficient to keep the stent-graft from migrating and to minimize endoleak. However, stent migration and endoleak may occur particularly in vessels that have irregular shapes or are shaped such that they exacerbate hemodynamic forces within the lumen. Stent migration refers to a stent graft moving from the original deployment site, usually in the direction of the blood flow. Endoleak (Type I) refers to the seepage of blood around the stent ends to pressurize the aneurismal sac or between the stent graft and the lumen wall. Stent graft migration may result in the aneurysmal sac being exposed to blood pressure again and increasing the risk of rupture. Endoleaks (of all types) occur in 10-25% of aneurysms treated with stent grafts. Some surgeons believe that endoleaks increase the risk of aneurysm expansion or rupture. Therefore, it would be desirable to have devices, compositions and methods that minimize post implantation stent graft migration and endoleak.

The blood vessel wall's blood-contacting lumen surface comprises a layer of endothelial cells. In the normal mature vessel the endothelial cells are quiescent and do not multiply. Thus, a stent graft carefully placed against the vessel wall's blood-contacting luminal surface rests against a quiescent endothelial cell layer. However, the normally quiescent endothelial cells lining the vessel wall, and in intimate contact with the stent graft luminal wall contacting surface, can be stimulated to proliferate. As these cells proliferate they will grow into and around the stent graft lining such that the stent graft becomes mechanically attached to the vessel lumen rather than merely resting against it.

Endothelialization has been observed to naturally occur in few human coronary stents within weeks of implantation. This natural endothelialization is not complete or consistent, however, and does not prevent the stent graft side effects of graft migration and endoleak. Methods to increase endothelialization are sought to improve clinical outcome after stent grafting.

Endothelialization may be stimulated by induced angiogenesis resulting in formation of new capillaries in the interstitial space and surface endothelialization. This has led to modification of medical devices with vascular endothelial growth factor (VEGF) and fibroblast growth factors 1 and 2 (FGF-1, FGF-2). The discussion of these factors is for exemplary purposes only, as those of skill in the art will recognize that numerous other growth factors have the potential to induce cell-specific endothelialization. The development of genetically-engineered growth factors is anticipated to yield more potent endothelial cell-specific growth factors. Additionally small molecule drugs may also induce endothelialization.

In one embodiment, a platelet gel effective to promote tissue growth into a stent graft is administered to a treatment site within a vessel lumen, either before, during or after stent graft implantation. Platelet gel compositions useful for promoting tissue growth into stent grafts, sealing stent graft to a vessel lumen and healing the aneurysm sac include, but are not limited to, platelet gel, autologous platelet gel, platelet rich plasma, platelet poor plasma, and thrombin, and combinations thereof. As used herein, “platelet plasma” refers to either or both of platelet poor plasma or platelet rich plasma.

Platelet gel is formed from plasma centrifugation products mixed with thrombin in the presence of calcium. Variable speed centrifugation of blood, preferably autologous blood, using devices such as, but not limited to, Medtronic Inc.'s Magellan™ Autologous Platelet Separation System results in the formation of platelet plasma, either platelet rich plasma (PRP) or platelet poor plasma (PPP). Platelet plasma contains sufficient fibrinogen to allow a fibrin gel to form when mixed with calcium and thrombin. Platelet rich plasma and PPP can be used in all embodiments using platelet gel as disclosed herein. In addition, PRP contains a high concentration of platelets that can aggregate for plugging, as well as release cytokines, growth factors or enzymes following activation by thrombin. PRP also contains WBC Fractions which may also contain multifunctional precursor cells or stem cells able to contribute to healing reactions in the aneurysmal sac. Some of the many factors released by the platelets and the white blood cells present that constitute PRP include platelet-derived growth factor (PDGF), platelet-derived epidermal growth factor (PDEGF), fibroblast growth factor (FGF), transforming growth factor-beta (TGF-β) and platelet-derived angiogenesis growth factor (PDAF). These factors have been implicated in wound healing by increasing the rate of collagen secretion, vascular in-growth and fibroblast proliferation.

Implantable medical devices, specifically stent grafts, are advantageously sealed to the vessel lumen using platelet gel. Platelet gel comprises platelet aggregates which help mechanically seal the stent graft to the lumen wall in addition to providing a rich source of growth factors. Briefly, following activation by thrombin, platelets release thromboxane A2, adenosine diphosphate and thrombin, factors that attract additional platelets to the developing clot. Once associated with the stent graft, platelet gel, with its rich composition of growth and healing factors, can promote the integration of the stent graft into the vessel wall. Enhanced healing and tissue in-growth from the surrounding vessel may lessen the chances for graft migration and endoleak. Additionally, drugs that inhibit pathological processes involved in aneurysm progression, such as, but not limited to inhibitors of matrix metalloproteinases, can be incorporated into the gel to enhance wound healing and/or stabilize and possibly reverse the pathology. Additional cells can be added as well: bone marrow derived cells, mesenchymal stem cells adipose derived stem cells or other stem cell fractions. Drugs that induce other positive effects at the aneurysm site, including but not limited to anti-inflammatory agents, can also be delivered by platelet gel and the methods described.

In one embodiment, platelet gel is formed on the stent graft prior to deployment. The stent graft can be coated sequentially or simultaneously with platelet plasma and thrombin, thereby forming the platelet gel on the stent graft prior to deployment. In another embodiment, the platelet gel is formed at the treatment site by using a delivery catheter to deliver the components (platelet plasma and thrombin) directly into the aneurysm site. Single- or multi-lumen catheters may be used to deliver the components of platelet gel substantially simultaneously or sequentially to the treatment site.

Because of the physical properties of platelet gel, it may be particularly useful in promoting endothelialization of vascular stent grafts. The platelet gel not only can coat the exterior surface of the stent graft but also fills the pores, inducing migrating cells into the stent graft fabric. As a result, engraftment of autologous endothelial cells will occur preferentially at those sites where platelet gel or it components were injected. Additionally the platelet gel may fill gaps between the stent graft outer wall and the inner lumen of the aneurysm sac further preventing endoleaks and providing structural support for weakened arterial walls within the aneurysm sac.

Some embodiments provide coatings for stent grafts that incorporate endothelialization factors other than platelet gel including, but not limited to growth factors and drugs.

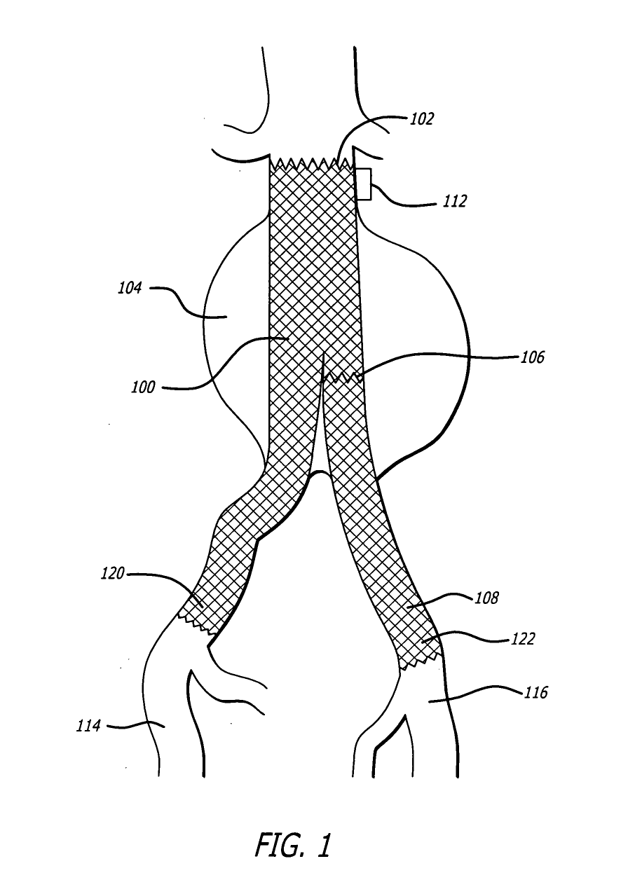

In some embodiments, the stent graft is provided “pre-loaded” into a deployment catheter and the platelet gel is applied to the stent graft directly prior to deployment. In another embodiment, platelet gel is applied directly to the treatment site approximately contemporaneous with stent graft deployment. In an exemplary stent deployment protocol to the site of an abdominal aortic aneurysm (FIG. 1), a vascular stent graft 100 is fully deployed through the left iliac artery 114 to an aneurysm site 104. Stent graft 100 has a proximal end 102 and an iliac leg 108 to anchor the stent graft in the right iliac artery 116. Stent graft 100 is deployed first by a first deployment catheter spanning the aneurysmal site 104 and iliac leg 108 is deployed by a second deployment catheter and are joined in an overlapping arrangement at overlap 106. Furthermore, after deployment, stent graft 100 contacts the blood vessel wall at least at sites 112, 120 and 122 to prevent leakage of blood into the aneurysm sac at these points. (The proximal end of the stent graft is the end nearest the heart by way of blood flow from the heart to the stent graft—while in the delivery system (catheter) the proximal end is the end nearest the operator and the distal end of the delivery system is the end holding the stent graft).

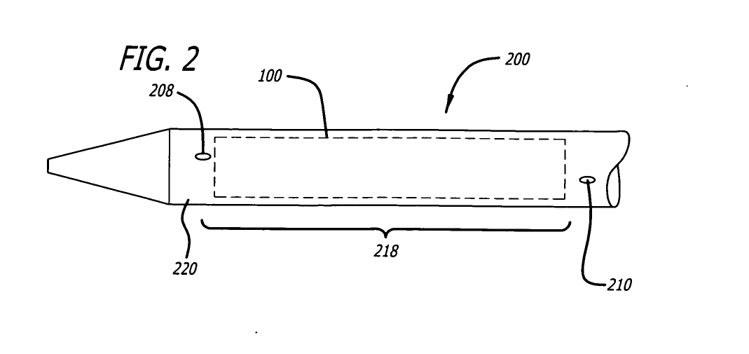

In one embodiment, a stent graft is pre-loaded into a delivery catheter such as that depicted in FIG. 2. Stent graft 100 is radially compressed to fill the stent graft chamber 218 in the distal end of delivery catheter 200. The stent graft 100 is covered with a retractable sheath 220. Delivery catheter 200 has two injection ports 208 and 210 for applying platelet plasma, thrombin or other compositions onto the abluminal surface of the stent graft prior to deployment. In this embodiment, a first component (platelet plasma or thrombin) is injected through injection port 208 to wet stent graft 100. A second component (platelet plasma or thrombin, whichever was not delivered as the first component) is next injected through injection port 210 to react with the first composition to form platelet gel on the abluminal surface of stent graft 100 within stent graft chamber 218. Stent graft 100 is then deployed to the treatment site as depicted in FIG. 1.

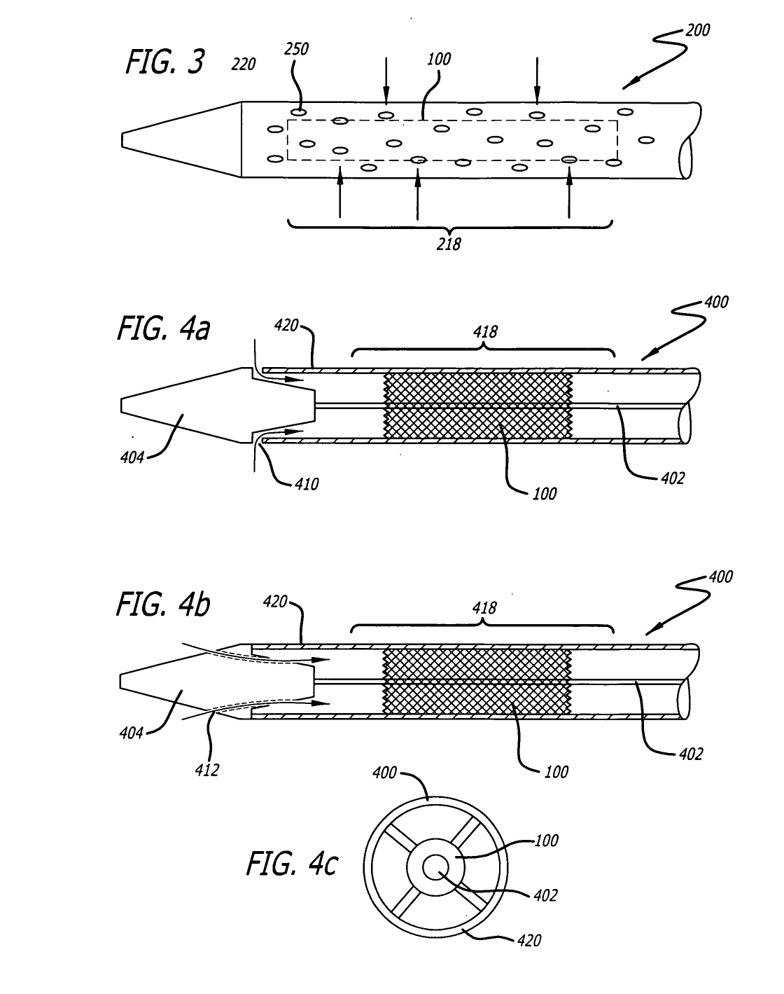

Another embodiment for coating the abluminal surface of a stent graft 100 within a delivery catheter 200 is depicted in FIG. 3. Retractable sheath 220 contains a plurality of holes 250 through which platelet plasma and thrombin can be sequentially or simultaneous applied to the abluminal surface of stent graft 100 compressed within stent graft chamber 218 prior to deployment.

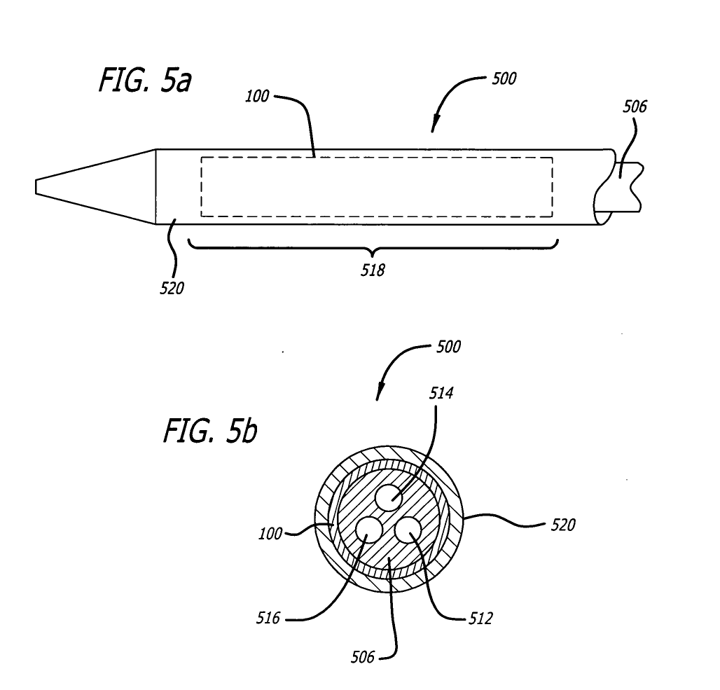

In yet another embodiment, platelet plasma and thrombin are applied to the abluminal surface of stent graft 100 within delivery catheter 400 as depicted in FIGS. 4a-c. Stent graft 100 is compressed into stent graft chamber 418 or delivery catheter 400 and held in its compressed state by sheath 420. A gap between the end of the sheath 420 and the mating portion of the taper tip 404 provides a passageway to deliver platelet plasma and thrombin to the stent graft while contained within the sheath 420. The platelet plasma and thrombin material is delivered to the gap and be sucked in through the gap or be pressurized to pass through the orifice created by the gap as suggested by the flow arrows 410 (FIG. 4a). The platelet plasma and thrombin can bathe the stent graft inside the sheath 420. A center guidewire lumen containing shaft 402 connects to the taper tip. As shown in FIG. 4b, an alternate method of delivering the platelet plasma and thrombin to the stent graft is by using a tapered tip 404 in which one or more (two dashed line passages are shown) passages for the passage of platelet plasma and thrombin from outside the delivery system to inside the delivery system to bathe or coat the constrained stent graft before delivery. Vacuum or pressure can be used to assist in the infusion of the platelet plasma or thrombin as previously discussed. The path of flow in FIG. 4b is depicted by arrows 412. FIG. 4c shows a possible cross section of the delivery system elements holding the stent graft compressed in a configuration where its outer surface is not in contact with the inner wall of the delivery catheter 400 so that the platelet plasma can bathe the outside portion of the stent graft. The covering holding the stent graft compressed will include passageways therethrough (such as a mesh) to allow the passage of fluid to the outside of the stent graft for coating or bathing.

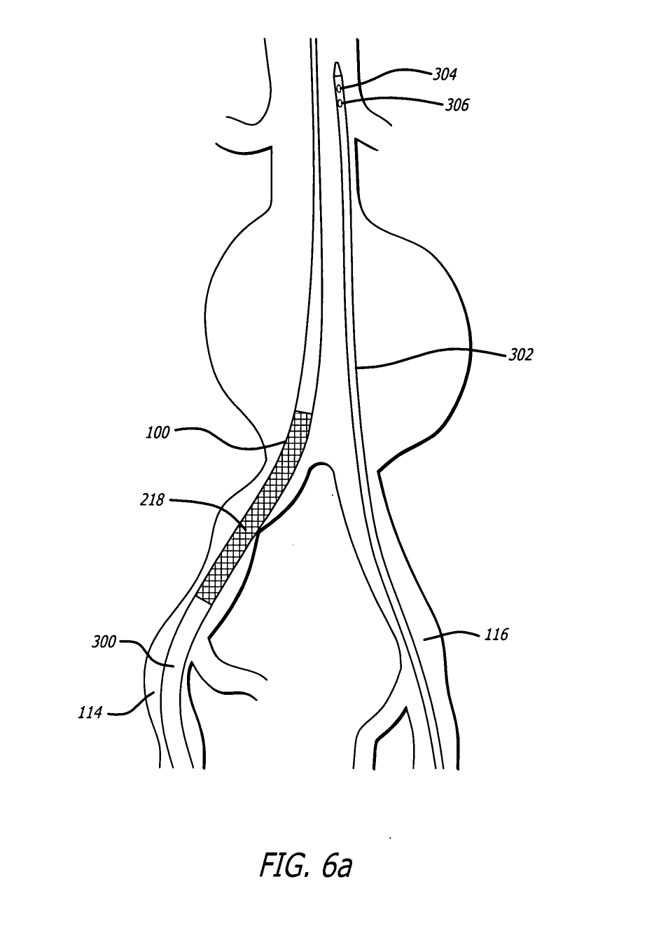

Alternatively, the luminal surface of stent graft 100 in delivery catheter 500 (FIG. 5a) is coated with platelet plasma and thrombin. Within the lumen of catheter 500 is a multilumen injection means 506 (FIG. 5b). Injection means 506 has a lumen 512 for delivery of the first component and a lumen 514 for delivery of the second component. An optional third lumen 516 is available for delivery of another composition such as, but not limited to, one or more drugs. The injection means 506 sequentially or simultaneously delivers the first component and the second component to the luminal surface of stent graft 100 prior to deployment. After coating of the stent graft with platelet gel, injection means 506 is removed from delivery catheter 500 and stent graft 100 is deployed to the treatment site as depicted in FIG. 1.

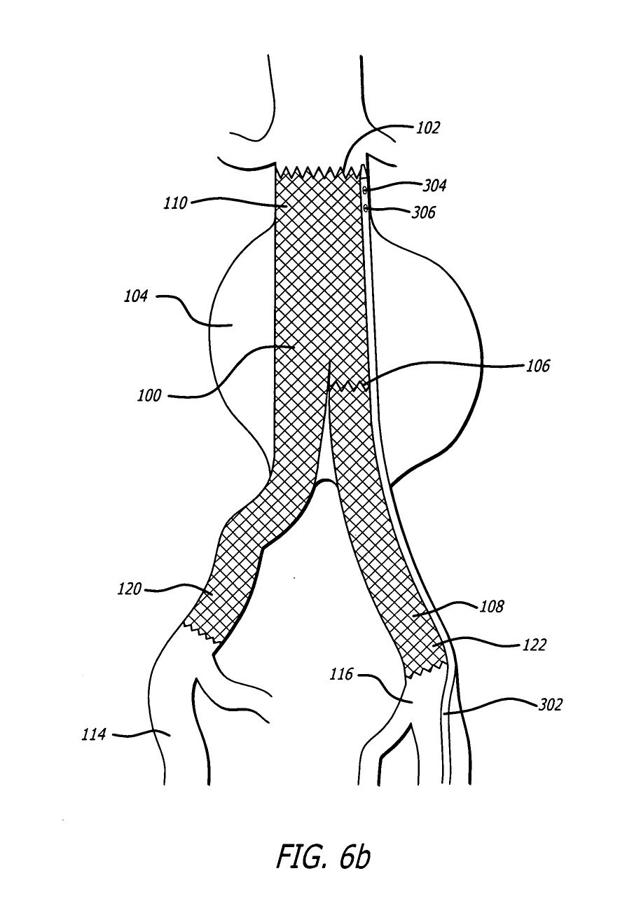

In another embodiment, the platelet composition is injected between the stent graft and the vessel wall during or after stent graft placement. As depicted in FIG. 6a, a stent graft 100 is radially compressed to fill the stent graft chamber 218 of stent delivery catheter 300 which is then deployed to the treatment site via the left iliac artery 114. A multilumen injection catheter 302 is also deployed to the treatment site through the right iliac artery 116. The multilumen injection catheter 302 can be a coaxial catheter with two injection lumens or a dual lumen catheter or alternatively a three lumen catheter if a guide wire lumen is required. Injection catheter 302 has injection ports 304 and 306 through which platelet plasma and thrombin may be delivered to a treatment site. In the first step of this deployment scheme (FIG. 6a), the stent delivery catheter 300 and the injection catheter 302 are deployed independently to the treatment site and the stent 100 is deployed. Delivery catheter 300 is removed and the iliac limb 108 is deployed as in FIG. 1 while the injection catheter 302 remains in place (FIG. 6b) with its injection ports 304 and 306 aligned with the proximal end 102 of stent graft 100. Thrombin and platelet plasma are injected approximately simultaneously between the vessel lumen wall and the stent graft at the proximal end 102 of stent graft 100 to form platelet gel 308 (FIG. 6c) to seal the proximal end 102 of stent graph 100 to the vessel wall at site 110. The injection catheter 302 is then retrieved. This same procedure can be repeated as necessary to apply platelet gel to the stent graft and/or luminal wall at other locations including, but not limited to, sites 110, 106, 120 and 122.

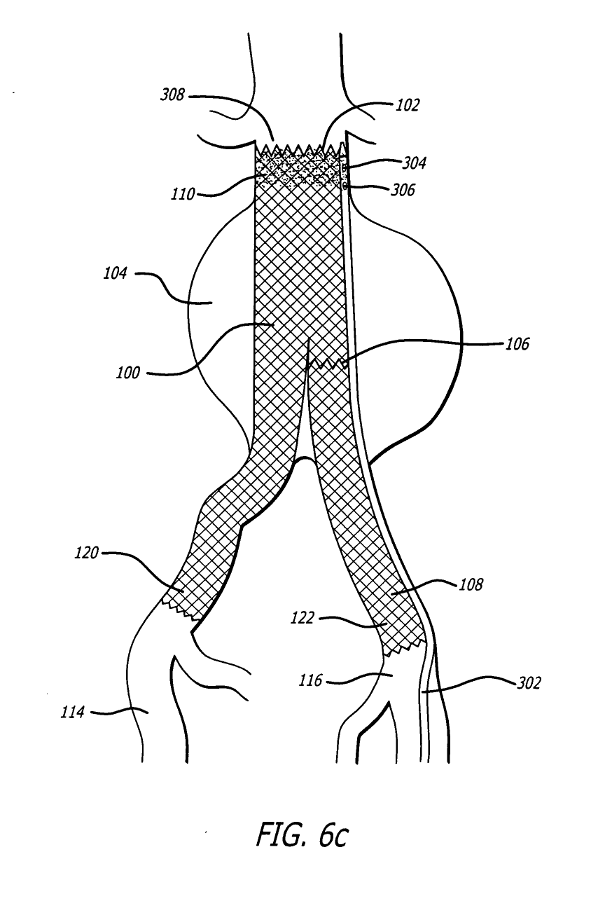

In another embodiment, platelet gel is delivered directly to the aneurysm sac. As previously described in FIG. 6a-c, in FIG. 7a, stent graft 100 is deployed. Injection catheter 302 is also deployed to the aneurysm sac through the right iliac artery 116. The injection catheter 302 can be a coaxial catheter with two injection lumens or a dual lumen catheter or alternatively a three lumen catheter if a guide wire lumen is required. Injection catheter 302 has injection ports 304 and 306 through which platelet plasma and thrombin may be delivered to the treatment site. Delivery catheter 300 is removed and the iliac limb 108 is deployed as in FIG. 6a-c while the injection catheter 302 remains in place with injection ports 304 and 306 in the aneurysm sac 104 (FIG. 7a). The iliac limb segment 108 of stent graft 100 seals the aneurysm sac at the distal end to the sealing site 122. Thrombin and platelet plasma are injected simultaneously between the vessel lumen wall and the stent graft within the aneurysm sac to form platelet gel 308 (FIG. 7b). An amount of PRP and thrombin necessary to produce enough platelet gel to fill the aneurysm sac is platelet plasma to the aneurysm sac. The injection catheter 302 is then retrieved.

In another embodiment, single lumen injection catheters can be used in the place of multilumen injection catheter 302. After the guide wire is retrieved from the lumen, platelet plasma and thrombin can be delivered to the treatment site sequentially through the same lumen of the single lumen injection catheter. In an alternate embodiment, more than one single lumen injection catheters can be deployed in each iliac artery with the distal ends of the catheters meeting in the aneurysm sac. Thrombin and platelet plasma can then each be injected through the single lumen injection catheters to form platelet gel in the aneurysm sac.

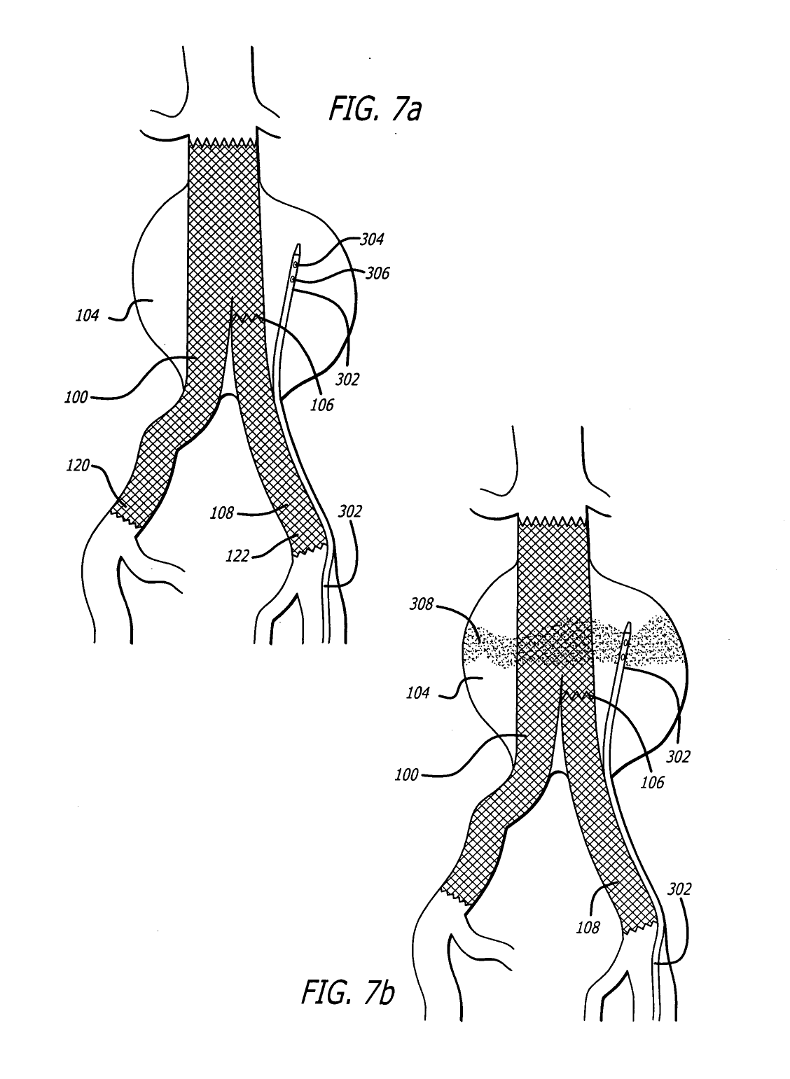

In an alternative embodiment, more than one injection catheter can be used to deliver platelet gel within the aneurysm sac (FIG. 8). As previously described in FIGS. 1 and 6, stent graft 100 is deployed to the treatment site via the left iliac artery 114 (FIG. 8a). Multiple single lumen or multilumen injection catheters 302 and 500 are also deployed to the aneurysm sac through the right iliac artery 116 and left iliac artery 114 (FIG. 8a). Injection catheters 302 and 500 have injection ports through which PRP and thrombin may be deposited. Delivery catheter 300 is removed and the iliac limb 108 is deployed as in FIGS. 1 and 6 while injection catheters 302 and 500 remain in place (FIG. 8b) with their injection ports 304 and 306 and 504 and 506 in aneurysm sac 104. The iliac limb segment 108 of stent graft 100 seals the aneurysm sac at the distal end at site 122. Thrombin and platelet plasma are injected substantially simultaneously between the vessel lumen wall and the stent graft within the aneurysm sac to form platelet gel 308 (FIG. 8c). An amount of platelet plasma and thrombin necessary to produce enough platelet gel to fill the aneurysm sac and seal the ends is determined radiographically by measuring the size of the aneurysm sac prior to surgery. The injection catheters 302 and 500 are then retrieved.

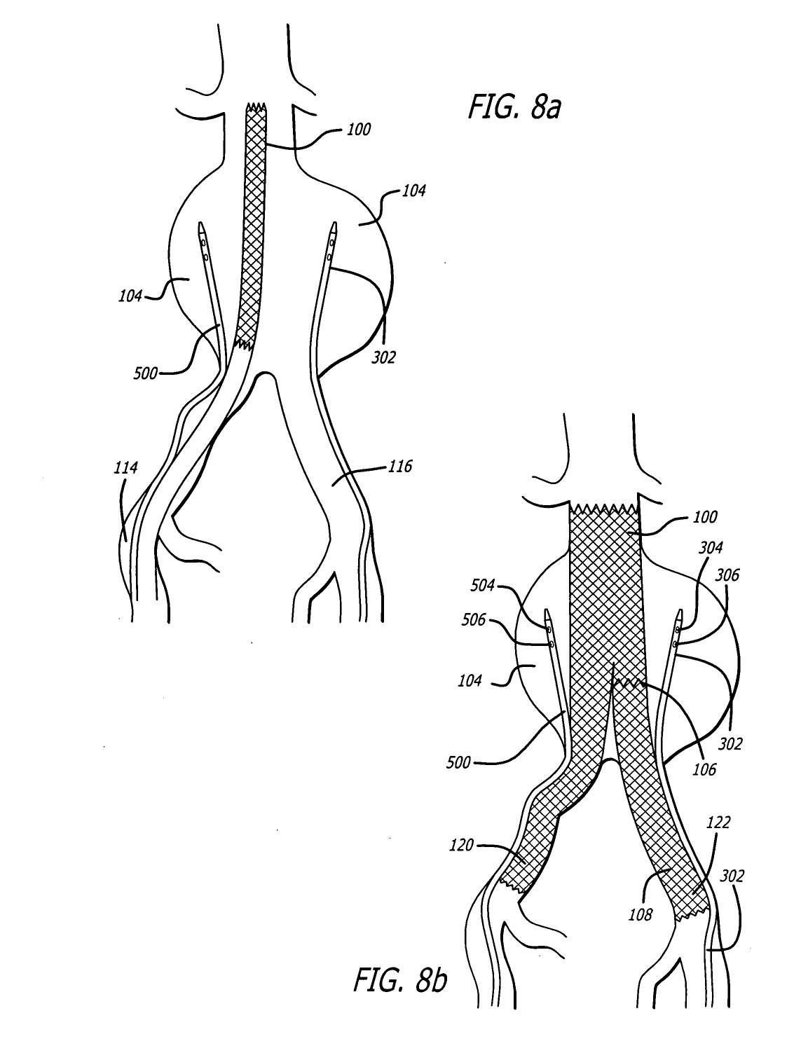

In yet another embodiment, platelet gel components are delivered to the aneurysm sac 104 by injecting the components through the wall of stent graft 100 (FIG. 9). Injection catheter 900 is advanced to the site of an already deployed stent graft 100 and needle 902 penetrates stent graft 100 to deliver platelet plasma and thrombin to the aneurysm sac 104 to form platelet gel 308. Injection catheter 900 can be a multi-lumen or single lumen catheter. If injection catheter 900 is single lumen, platelet plasma and thrombin can be delivered sequentially from the same or different catheters.

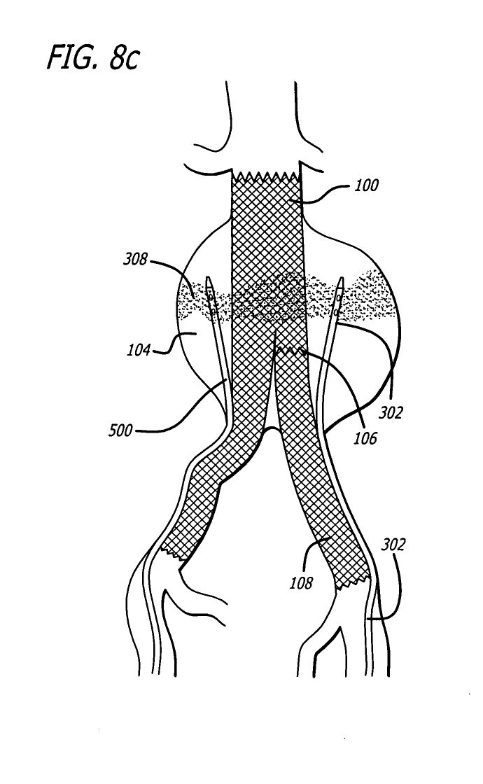

In another embodiment, platelet gel components are delivered to the aneurysm sac 104 by translumbar injection (FIG. 10). Injection means 920, such as but not limited to a syringe, is directed, under radiographic or echographic guidance, to the aneurysm sac where stent graft 100 and iliac leg 108 have already been deployed. Injection means 920 delivers platelet plasma and thrombin to the aneurysm sac 104 to form platelet gel 308. Injection means 920 can have a single lumen or multiple lumens. If injection means 920 is single lumen, platelet plasma and thrombin can be delivered sequentially from the same or different injection means.

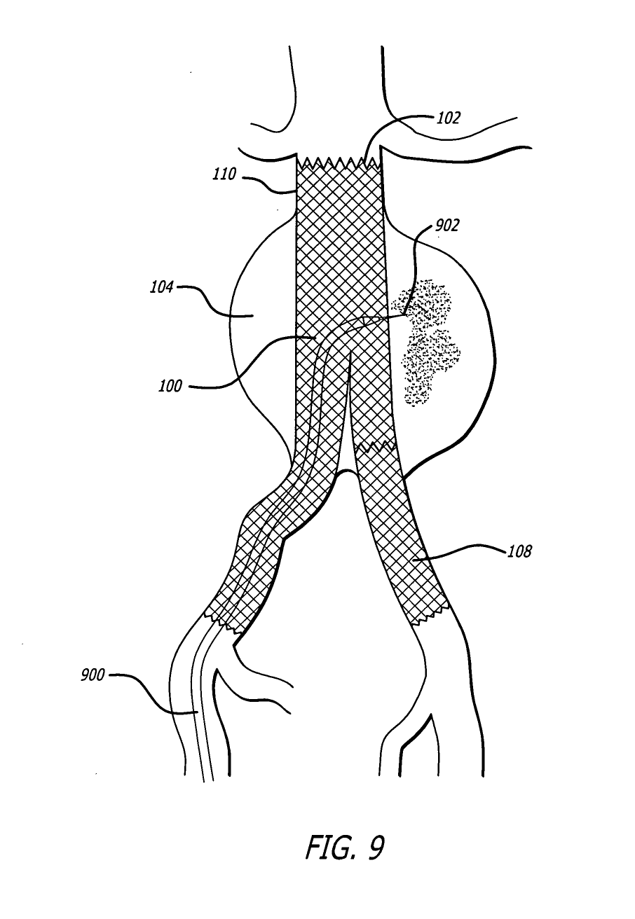

In yet another embodiment, depending on aneurysm location and stent placement, a collateral artery can be used to access the luminal wall-contacting surface of a deployed stent graft (FIG. 11). For example, and not intended as a limitation, stent graft 100 may be deployed such that the proximal end 102 is in the abdominal aorta 154 near, but below renal artery. After deployment of stent graft 100, the deployment catheter is removed and an injection catheter 302 is advanced up the aorta past the aneurysm sac 104 to the superior mesenteric artery 150. The injection catheter 302 is then advanced through the superior mesenteric artery 150 and down into the inferior mesenteric artery 152 where it originates at the aorta within aneurysm sac 104. The platelet plasma and thrombin are then injected into the aneurysm sac 104 to form platelet gel 308 therein. Alternatively, if inferior mesenteric artery 152 originates adjacent to aneurysm sac 104 but within the aorta occupied by stent graft 100, platelet plasma and thrombin may be delivered to any site between stent graft 100 and the vessel wall accessible from inferior mesenteric artery 152.

Once the platelet gel has been administered to the stent graft/vessel lumen interface or aneurysm sac, endothelial cell growth will be activated and endothelial cells will proliferate and adhere to the stent graft (a condition or process referred to herein after as “tissue in-growth” or endothelialization) thus anchoring the stent graft securely to the vessel lumen and preventing stent graft migration. Moreover, tissue in-growth will also provide a seal between the luminal wall contacting surface of stent graft 100 at its proximal end or other locations at risk for endoleak including, but not limited to, sites 106, 110, 112, 120, and 122.

The following examples are meant to illustrate one or more embodiments and are not meant to limit its scope to that which is described.

### EXAMPLE 1

### Properties of Platelet Rich Plasma

Aliquots of human peripheral blood (30-60 mL) are passed through the Magellan™ Autologous Platelet Separation System (the Magellan™ system) and the concentrated, platelet-rich plasma fraction (PRP) assayed for platelets (PLT), white blood cells (WBC) and hematocrit (Hct) (Table 1). The Magellan™ system concentrated platelets and white blood cells six-fold and three-fold respectively.

TABLE 1
Blood cell yields after passing through the Magellan ™ system.

| Mean ± SD | | | |
|---|---|---|---|
| n = 19 | Initial Blood | PRP | Yield |
| PLT (×1000/μL) | 220.03 ± 48.58 | 1344.89 ± 302.00 | 6.14 ± 0.73 |
| WBC (×1000 μ/L) | 5.43 ± 1.43 | 17.04 ± 7.01 | 3.12 ± 0.90 |
| Hct (%) | 38.47 ± 2.95 | 6.81 ± 1.59 | |

Cell Yield = cell count in PRP/cell count in initial blood = [times baseline]

Additionally, PRP was assayed for levels of the endogenous growth factors platelet-derived growth factor (PDGF), transforming growth factor-beta (TGF-β), basic fibroblast growth factor (bFGF), vascular endothelial growth factor (VEGF), and endothelial growth factor (EGF). As a result of increased platelet and white blood cell counts in PRP, increased concentrations of growth factors were found.

TABLE 2
Growth Factor Content of Blood and PRP

| Mean ± SD; n = 9 | Initial Blood | PRP |
|---|---|---|
| PDGF-AB (ng/mL) | 10.2 ± 1.4 | 88.4 ± 28.8 |
| PDGF-AA (ng/mL) | 2.7 ± 0.5 | 22.2 ± 4.2 |
| PDGF-BB (ng/mL) | 5.8 ± 1.4 | 57.8 ± 36.6 |
| TGF-β1 (ng/mL) | 41.8 ± 9.5 | 231.6 ± 49.1 |
| bFGF (pg/mL) | 10.7 ± 2.9 | 48.4 ± 25.0 |
| VEGF (pg/mL) | 83.1 ± 65.5 | 597.4 ± 431.4 |
| EGF (pg/mL) | 12.9 ± 6.2 | 163.3 ± 49.4 |

### EXAMPLE 2

### Platelet Gel Generation

Platelet gel is generated from the PRP fraction produced in the Magellan system by adding thrombin and calcium to activate the fibrinogen present in the PRP. For each approximately 7-8 mL of PRP, approximately 5000 units of thrombin in 5 mL 10% calcium chloride are required for activation. Platelet gel is formed immediately upon mixing of the activator solution with the PRP. The concentration of thrombin can be varied from approximately 1-1,000 U/mL, depending on the speed required for setting to occur. The lower concentrations of thrombin will provide slower gelling times.

### EXAMPLE 3

### Effects of Platelet Gel on Cell Proliferation

A series of in vitro experiments were conducted evaluating the effect of released factors from platelet gel on the proliferation of the human microvascular endothelial cells, human coronary artery smooth muscle cells and human dermal fibroblasts. Primary cell cultures of the three cell types were established according to protocols well known to those skilled in the art of cell culture. For each cell type, three culture conditions were evaluated. For platelet gel cultures, platelet gel was added to cells in basal medium. A second group of cells were cultured in growth medium. Growth medium is the standard culture medium for the cell type and contains optimal growth factors and supplements. The control cultures contain cells cultured only in basal medium which contains no growth factors.

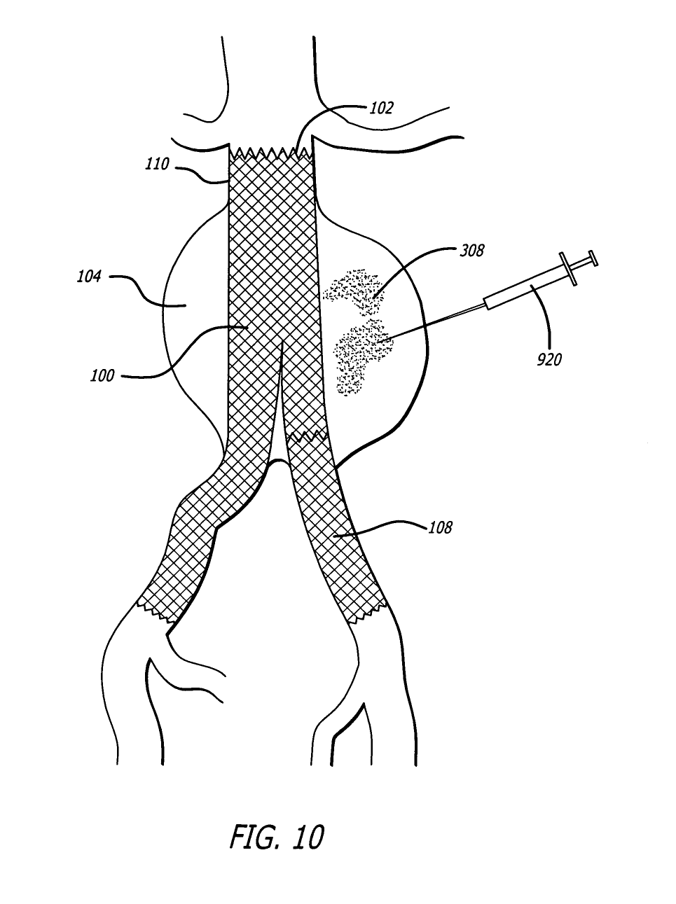

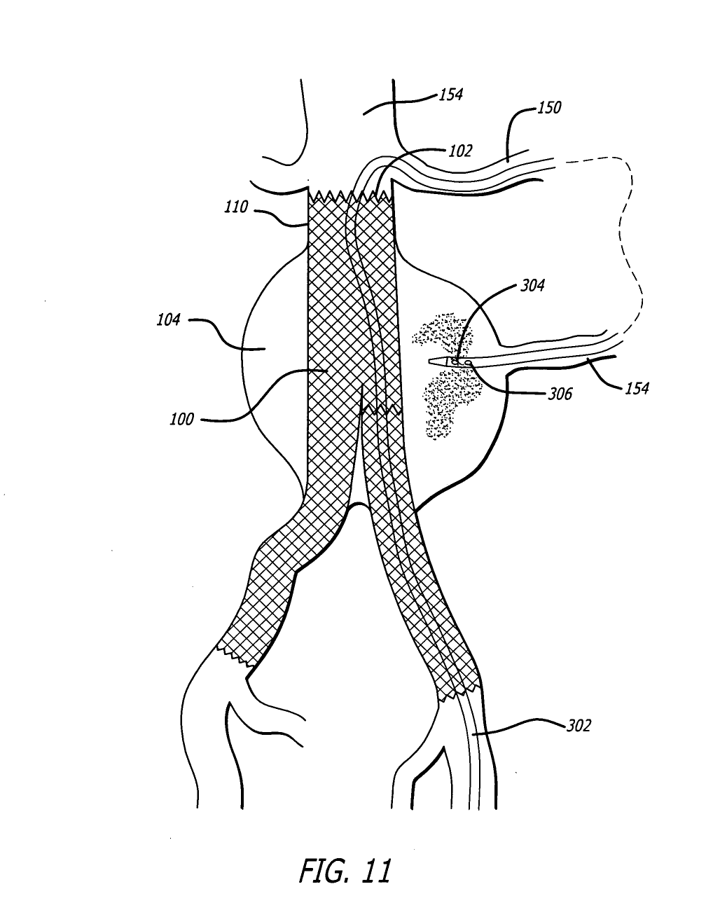

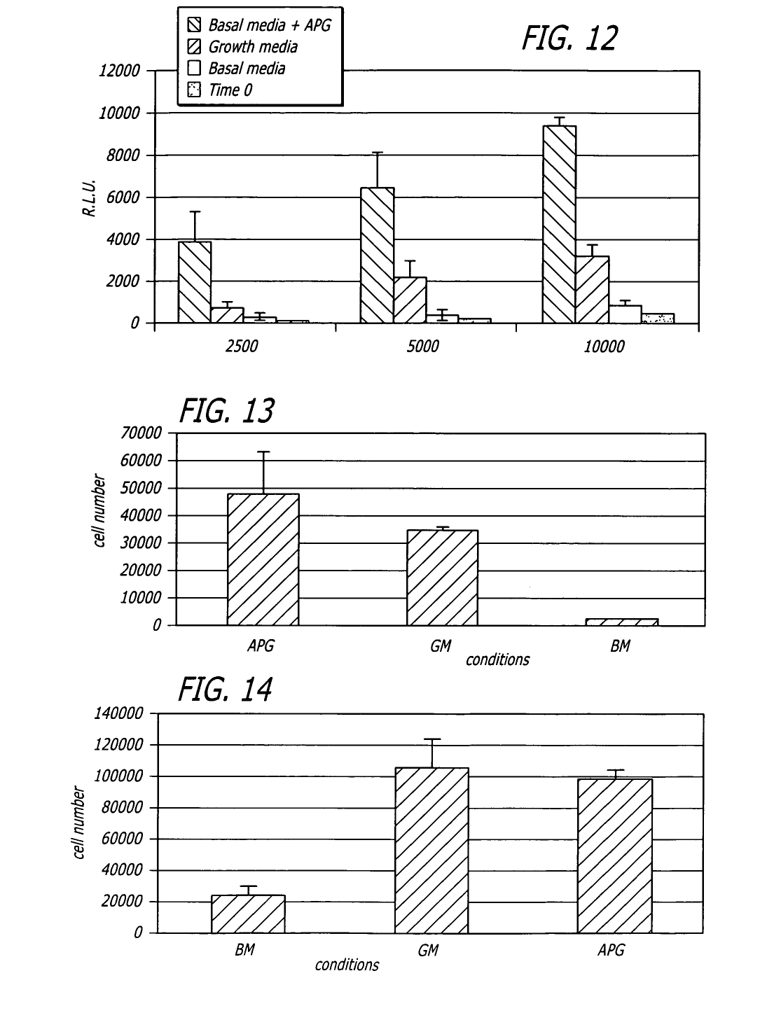

Platelet gel had a significant growth effect on human coronary artery smooth muscle cells after five days of culture (FIG. 12), human microvascular endothelial cells after four days of culture (FIG. 13) and on human dermal fibroblasts after five days of culture (FIG. 14).

### EXAMPLE 4

### Effect of Platelet Poor Plasma on Human Dermal Fibroblast Growth

In addition to the platelet-rich plasma fraction, the Magellan system generates a platelet-poor plasma (PPP) fraction as well. This PPP fraction was further processed by centrifuging at 10,000×g for 10 minutes. The PPP fractions were then activated with the CaCl₂/thrombin activator solution used in the APG generation. Human dermal fibroblasts were then cultured in basal medium containing PRP gel or PPP gel. Culture conditions for proliferation of human dermal fibroblasts are well known to those of ordinary skill in the art of cell culture.

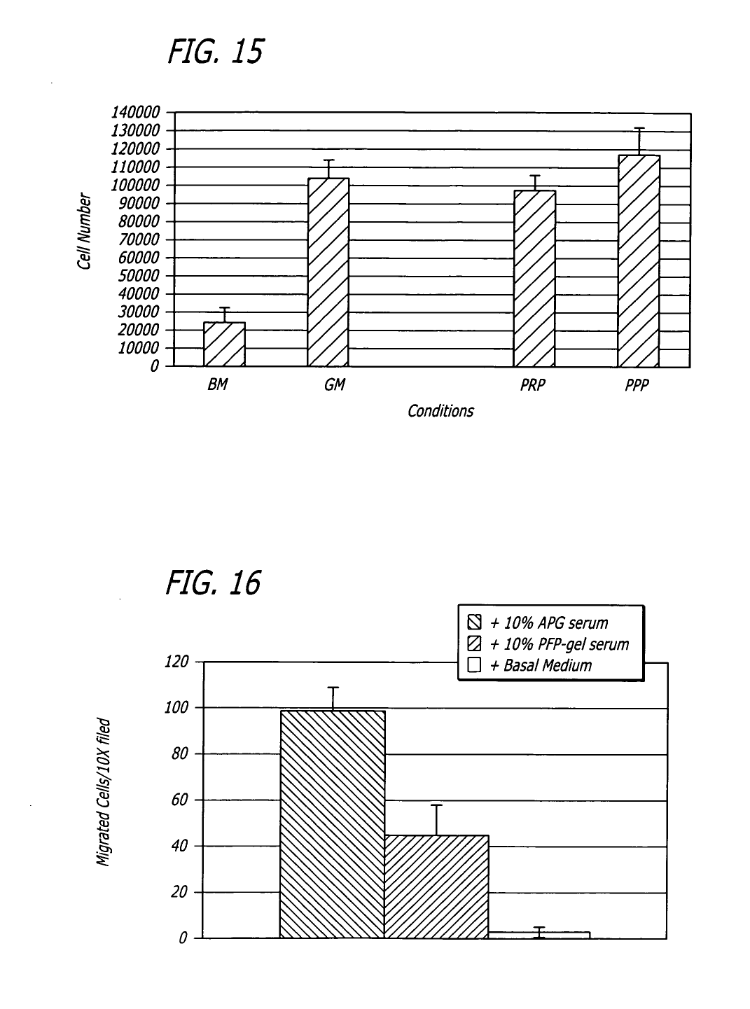

Human dermal fibroblasts cultured in the presence of PPP gel proliferated to a similar extent as those cultured in the presence of PRP gel (FIG. 15).

### EXAMPLE 5

### Effect of Platelet Gel on Endothelial Cell Migration

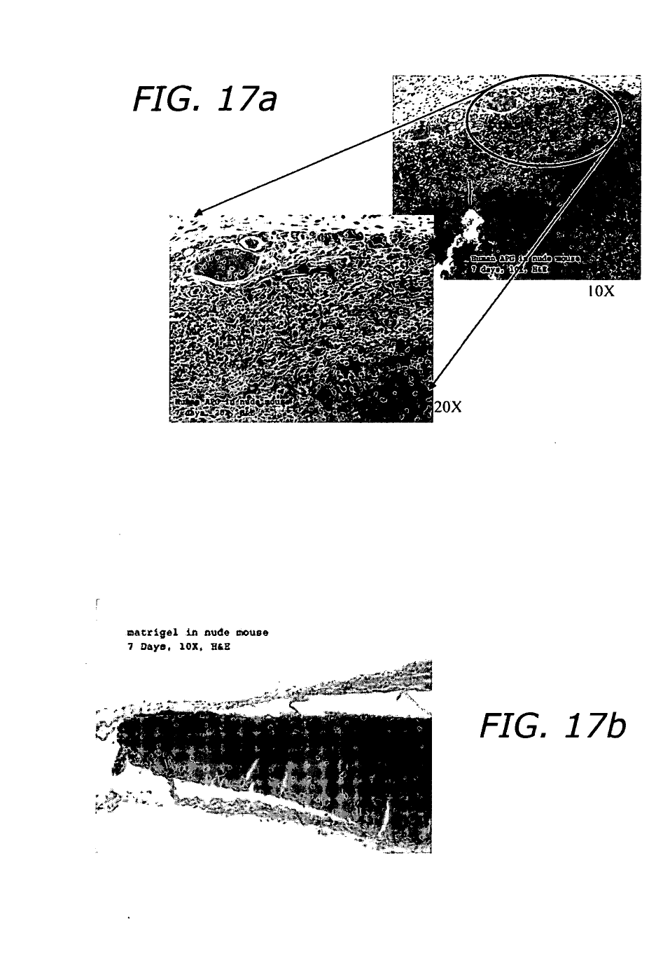

Human microvascular endothelial cell migration was performed in a Boyden chemotaxis chamber which allows cells to migrate through 8 μm pore size polycarbonate membranes in response to a chemotactic gradient. Human microvascular endothelial cells (5×10⁵) were trypsinized, washed and resuspended in serum-free medium (DMEM) and 400 μL of this suspension was added to the upper chamber of the chemotaxis assembly. The lower chamber was filled with 250 μL serum-free DMEM containing either 10% platelet gel, 10% platelet-free plasma (PFP) or DMEM alone. After a pre-determined amount of time, the filters were removed and the cells remaining on the upper surface of the membrane (cells that had not migrated through the filter) were removed with a cotton swab. The membranes were then sequentially fixed, stained and rinsed to enable the visualization and quantification of cells that had migrated through the pores to the other side of the membrane. Platelet gel induced significantly more migration in human microvascular endothelial cells than either PFP or basal medium (FIG. 16).

### EXAMPLE 6

### Effect of Platelet Gel on Neovascularization in Athymic Mice

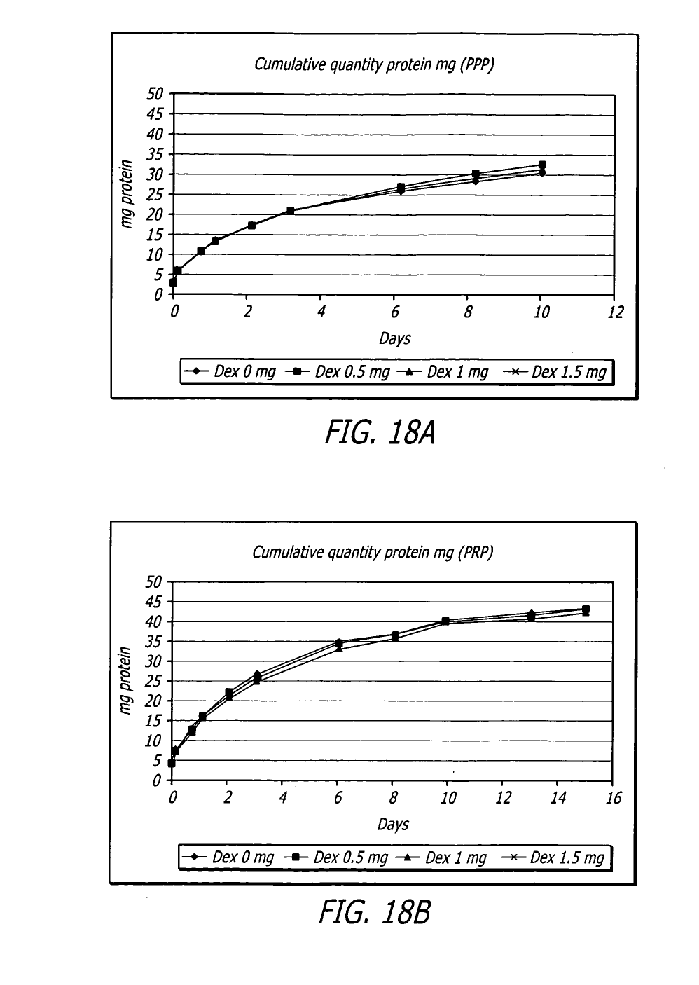

Platelet gel was injected subcutaneously in nude (athymic) mice to determine if the platelet gel is detectable and retrievable after a seven day implantation period. Athymic mice were injected with 500 μL of either platelet gel or an inert Matrigel® control. Each animal was injected with Matrigel® in the left flank and platelet gel in the right flank. After seven days the implants and the surrounding tissue were subjected to histological analysis (FIG. 17a-b). In the area of the Matrigel® control implant there was minimal reaction to the material and a very thin capsule of loose connective tissue rimmed the mass (FIG. 17a). In the area of the platelet gel implant, the platelet gel was deeply infiltrated by spindle shaped cells (fibroblasts and macrophages) along with moderate numbers of neutrophils (FIG. 17b). The entire mass was rimmed by a thick layer of fibrovascular tissue and the tissue showed significant neovascularization.

### EXAMPLE 7

### Loading of Bioactive Agents into Platelet Gels

Gels were formed in 5 mL polypropylene tubes by adding a selected bioactive agent in a quantity of between 5 μL and 15 μL, about 50 μL of thrombin and making the mixture up to 500 μL with PRP or PPP. The contents of the tubes were mixed and allowed to stand for 15 minutes at room temperature. After 15 minutes, 4 mL of sterile phosphate buffered saline was added to the gels. The tubes were then closed and releases were performed at 37° C. In order to refresh the gels, at designated times, 3 mL of release buffer, containing the dexamethasome or doxcyline, was removed and the gel was refreshed with the same quantity of refresh buffer. After the refresh buffer was removed after a final refreshing, 500 μL of drug-loaded gel remained.

The following data were generated for variations of two drugs, which are known matrix metalloproteinase inhibitors (Table 3).

TABLE 3

| | | | Max Loading |
|---|---|---|---|
| Bioactive Agent | Properties | Max Solubility | in Gels |
| Dexamethasome | Hydrophobic | 100 mg/mL in DI | 1.5 mg |
| Phosphate | | water | (3 mg/mL) |
| Dexamethasome | Hydrophobic | 50 mg/mL in ethanol | 0.5 mg |
| Acetate | | | (1 mg/mL) |
| Doxycline | Hydrophilic | 400 mg/mL in DI | 0.5 mg |
| Hydrochloride | | water | (1 mg/mL) |
| Doxycline | Hydrophobic | >100 mg/mL in | 0.5 mg |
| Monohydrate | | DMSO | (1 mg/mL) |

DI = deionized water;
DMSO = dimethylsulfoxide

### EXAMPLE 8

### Release of Bioactive Agents from Platelet Gels

Protein-free platelet gels for high performance liquid chromatography (HPLC) detection of bioactive agent were prepared using acetonitrile (ACN) protein precipitation. Each platelet gel loaded with a bioactive agent to be evaluated was diluted with ACN to a 3:1 ACN to sample ratio. The mixture was vortexed, then centrifuged at 5500 G for 10 minutes at 4° C. The supernatant was then decanted into an HPLC vial containing HPLC buffer consisting of 1.104 g/L NaH₂PO₄.H₂O and 0.89 g/L NaH₂PO₄.2H₂O. Concentrations of bioactive agents were then determined by HPLC by methods known by those skilled in the art.

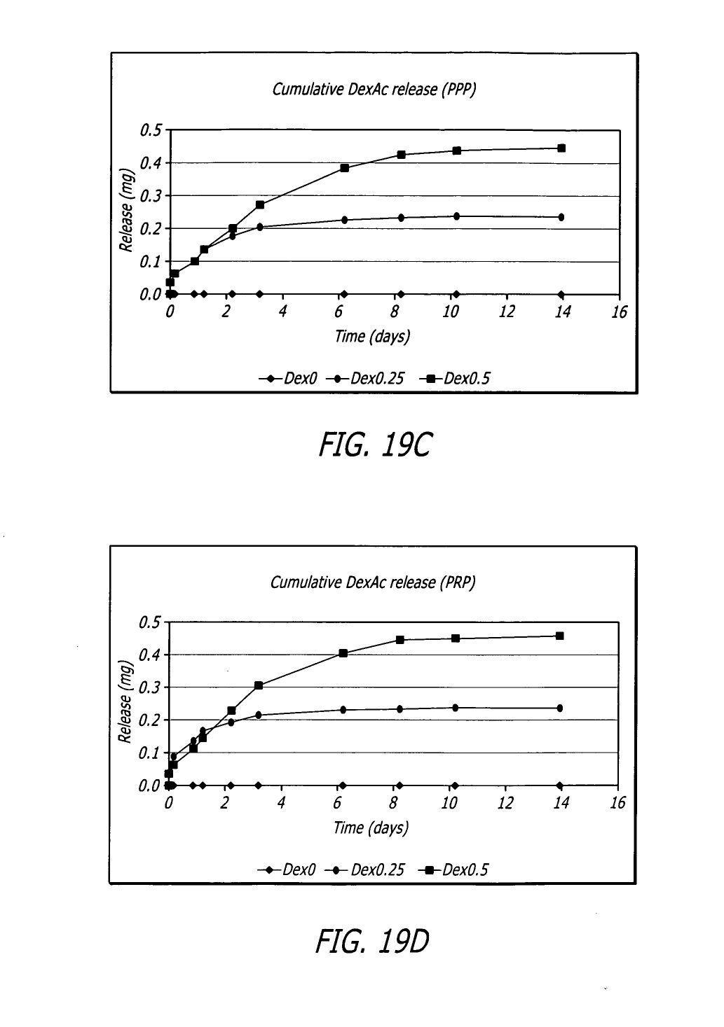

Results showed dose dependent release of bioactive agents and similar release profiles from both PPP (FIGS. 19A, 19C) and PRP (FIG. 19B, 19D) gels. Dexamethasome acetate (FIGS. 19C, 19D) had a lower recovery than that of dexamethasome phosphate (FIGS. 19A, 19B).

### EXAMPLE 9

### Protein Release from Platelet Gels

A colorimetric assay (Bio-RAD DC Protein Assay) was used for protein detection. Briefly, in this assay proteins react with an alkaline copper tartrate solution and Folin reagent to generate a yellow color, which is then measured by spectrometry. Standards of BSA were prepared for each experiment (0 to 1.6 mg/ml in PBS).

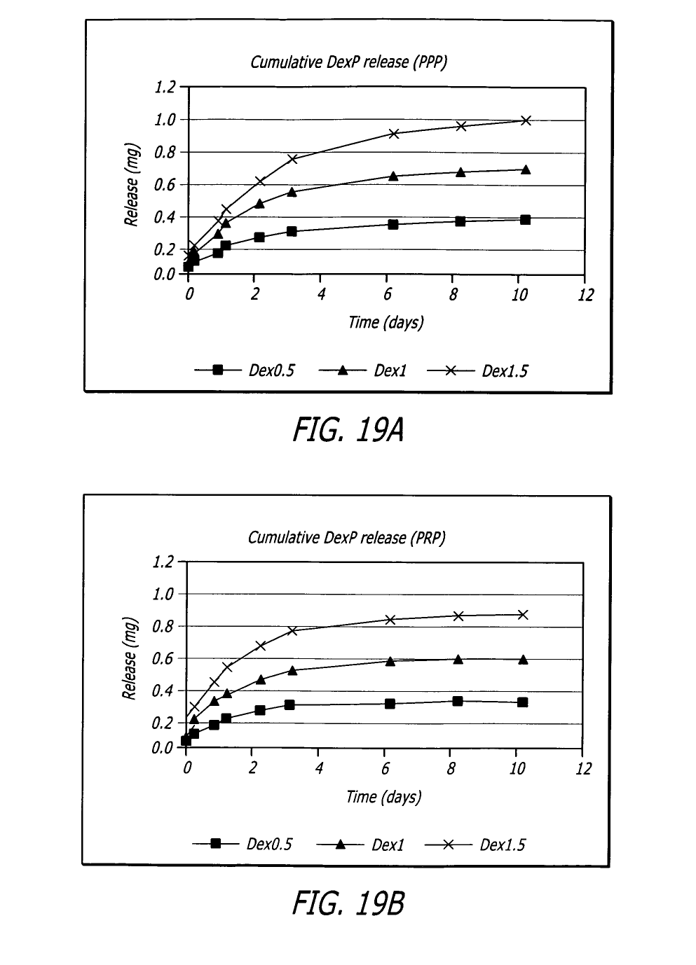

Results indicate a constant release of protein over time. Protein presence in the solution is as a result of protein release as well gradual degradation of the gel. Due to the higher platelet count, and therefore higher concentrations of growth factor release, higher protein presence in the release solution was observed with the PRP gels (FIG. 18B) than with the PPP gels (FIG. 18A). The presence of the drug in the gels (0.5 mg to 1.5 mg) did not impair release of protein.

### EXAMPLE 10

### Stability of Bioactive Agents in Platelet Gels

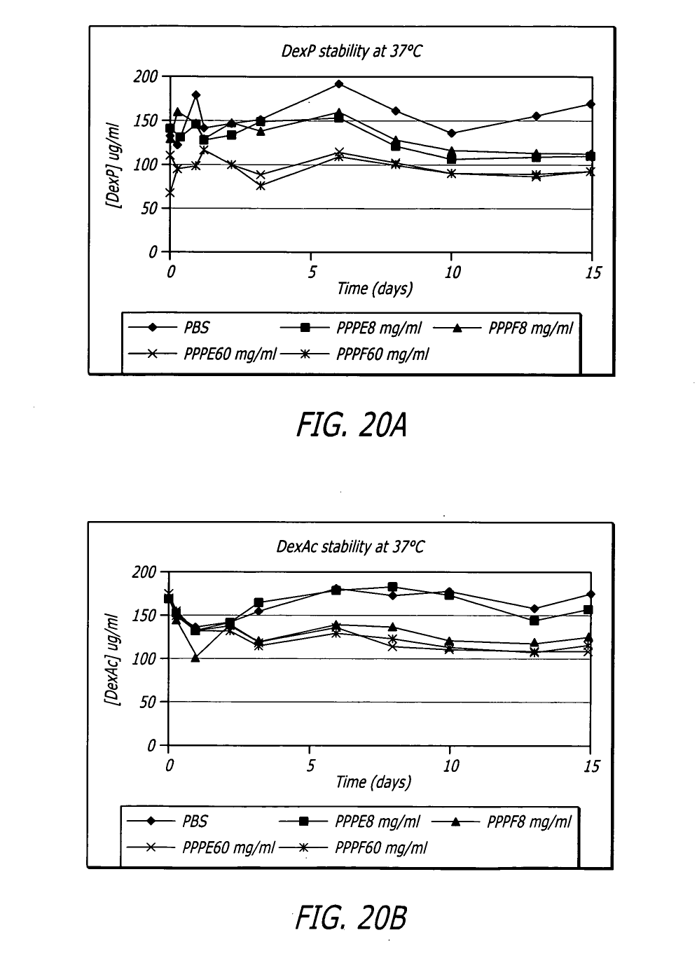

Drug degradation was determined for platelet gels loaded with dexamethasome. Literature reports a plasma half-life for dexamethasome of about 3-4 hours and a biological half-life of about 36-72 hours. In the case of the current platelet gels, dexamethasome phosphate had a stability in PBS up to 14 days. In diluted PPP (8 mg/mL protein), there was an initial dexamethasome loss, but levels remained stable thereafter. In concentrated PPP, (about 60 mg/mL protein), greater dexamethasome loss was observed from early time points. Dexamethasome acetate was slightly more stable under all conditions tested (FIGS. 20A and 20B).

### EXAMPLE 11

### Effect of Bioactive Agents on Growth Factor Properties of Platelet Gels

Gels were formed in microfuge tubes by two different methods. One set of PRP gels were formed by adding drugs and thrombin to the PRP and mixing. A second set of gels was formed by adding drugs to the PRP, mixing, and letting the resulting mixture sit for 30 minutes. After 30 minutes, thrombin was added and the resulting solution was mixed. Both sets of gels were then allowed to stand for 20 minutes at room temperature. Gels were then disrupted within their respective tubes and subsequently centrifuged at 20,000 G for 5 minutes to express the serum. Then, the supernatant (growth factor enriched serum) was removed and serum samples were tested for effects on cell proliferation and growth factor profiles.

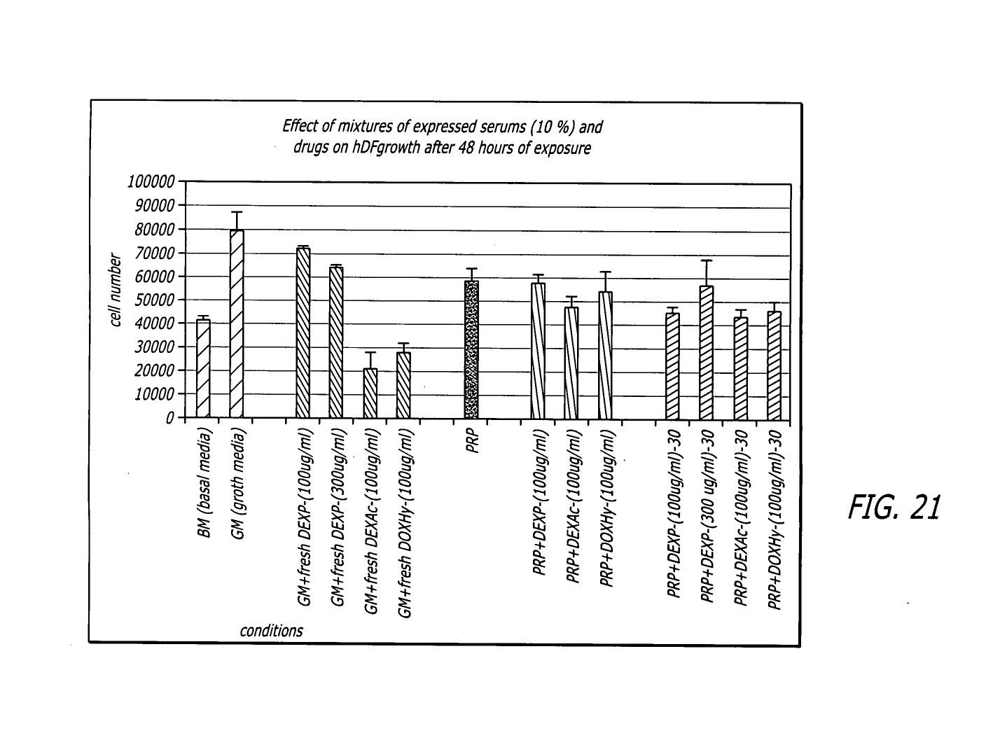

The presence of drugs in the gels did not alter the effects of PRP on fibroblast proliferation (FIG. 21). In addition, growth factors from PRP gels reversed the negative effects of dexamethasome acetate and doxcyline. Dexamethasome phosphate in particular had no effect on growth factor concentrations. Dexamethasome acetate resulted in lower levels with some growth factors. Doxycline consistently resulted in lower growth factor concentrations.

### EXAMPLE 12

### Bioactive Agent Containing Microspheres

Lactic acid/glycolic acid microspheres were produced by Innocore Technologies, BV (Groningen, Netherlands). The microspheres were loaded with 12.8% doxycline. The microspheres had a diameter size distribution (n=25) of 101.95 μm (standard deviation of 46.22 μm). The microspheres were designed for a 14 day release of doxycline.

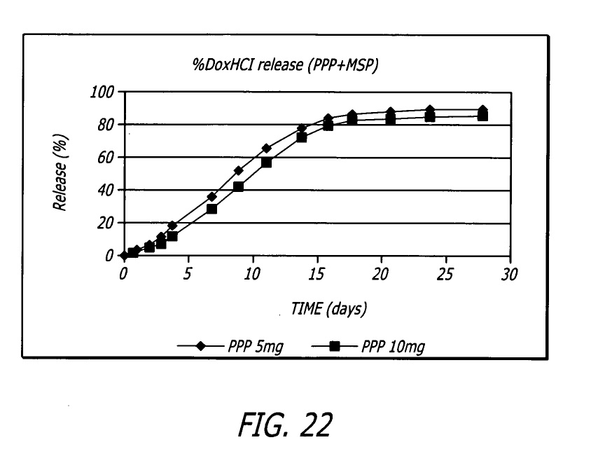

PPP gels were loaded with 5 and 10 mg of doxcyline loaded microspheres. Drug release was monitored from the PPP gels. A slight delay in the release of doxcyline was observed at early time points, but an 80% release was observed by day 14 (FIG. 22). Further experimentation demonstrated that 100 mg of microspheres could be loaded into 500 μL of gel.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification are approximations that may vary depending upon the desired properties sought to be obtained according to the present invention. At the very least, each numerical parameter should at least be construed in light of the number of reported significant digits and by applying ordinary rounding techniques. Notwithstanding that the numerical ranges and parameters setting forth are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing embodiments according to the invention are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate embodiments and not to pose a limitation on possible alternatives. No language in the specification should be construed as indicating any element to be essential.

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, of course, variations on these described embodiments will become apparent to those of ordinary skill in the art upon reading the foregoing description.

Furthermore, numerous references have been made to patents and printed publications throughout this specification. Each of the above-cited references and printed publications are individually incorporated herein by reference in their entirety.

What is claimed is:

1. A stent graft comprising:
an abluminal surface;
a luminal surface; and
platelet gel on at least one of said abluminal and said luminal surfaces, wherein said platelet gel further comprises a bioactive agent and wherein said abluminal surface of said stent graft is coated with platelet gel prior to deployment by depositing platelet plasma and thrombin on said stent graft compressed within a stent graft chamber of a stent deployment catheter.

2. The stent graft according to claim 1 wherein said platelet gel is applied directly to said stent graft compressed within a stent deployment catheter.

3. The stent graft according to claim 1 wherein said platelet gel comprises thrombin and platelet plasma.

4. The stent graft according to claim 3 wherein said platelet plasma comprises at least one of platelet rich plasma or platelet poor plasma.

5. The stent graph according to claim 3 wherein said platelet plasma and/or said thrombin are autologous.

6. The stent graft according to claim 1 wherein said platelet gel further comprises one or more bioactive agents selected from the group consisting of small molecules, peptides, proteins, hormones, DNA or RNA fragments, cells, genetically engineered cells, genes, cell growth promoting compositions and matrix metalloproteinase inhibitors.

7. A method for providing a stent graft and platelet gel to a treatment site comprising:
delivering a stent graft to an aneurysm site; and
delivering to the abluminal surface of said stent graft thrombin and platelet plasma such that platelet gel is formed between said abluminal surface of said stent graft and the blood vessel wall.

8. The method according to claim 7 wherein said platelet gel substantially fills said aneurysm sac.

9. The method according to claim 7 wherein said platelet plasma comprises at least one of platelet rich plasma and platelet poor plasma.

10. The method according to claim 7 wherein said thrombin and/or said platelet plasma are autologous.

11. The method according to claim 7 wherein said platelet gel further comprises one or more bioactive agents is selected from the group consisting of small molecules, peptides, proteins, hormones, DNA or RNA fragments, cells, genetically engineered cells, genes, cell growth promoting compositions and matrix metalloproteinase inhibitors.

12. The method according to claim 7 further comprising:
advancing a stent deploying catheter containing a stent graft to a treatment site;
advancing at least one injection catheter containing at least one component of platelet gel to said treatment site;
deploying said stent graft at said treatment site; and
applying said components of said platelet gel from said at least one injection catheter to said inner lumen of said blood vessel at said treatment site to form platelet gel; such that said abluminal surface of said stent graft engages said platelet gel and said blood vessel luminal surface contacts said platelet gel at said treatment site.

13. The method according to claim 12 wherein the step of applying said components includes applying cell selected from among a list consisting of: stem cells, adipose stem cells, mesenchymal stem cells, and cells from bone marrow.

14. The method according to claim 7 wherein said injection catheter is selected from the group comprising single lumen injection catheter and multilumen injection catheter.

15. The method according to claim 7 wherein a first of said at least one injection catheter reaches said treatment site through a different route than a second of said at least one injection catheter.

16. The method according to claim 7 wherein said first of said at least one injection catheter reaches said treatment site through a blood vessel bisecting the treatment site thereby delivering said cell growth promoting composition directly to the aneurysm sac.

17. The method according to claim 16 wherein said treatment site is selected from the group consisting of the area where the proximal end of the stent graft contacts the vessel lumen wall, the junction between a stent graft and an iliac limb section, the aneurysm sac, and combinations thereof.

18. The method according to claim 7 wherein said thrombin is of bovine origin.

19. The method according to claim 7 wherein said thrombin is of recombinant human origin.

20. A method for providing a stent graft and platelet gel to aneurysm site comprising:
loading a stent graft into a delivery catheter, said stent graft comprising an abluminal surface and a luminal surface;
applying to at least one of said abluminal surface and said luminal surface, thrombin and platelet plasma to form platelet gel on said stent graft within said delivery catheter;
advancing said deployment catheter to said aneurysm site; and
deploying said stent graft at said aneurysm site.

21. The method according to claim 20 wherein said platelet plasma comprises at least one of platelet rich plasma or platelet poor plasma.

22. The method according to claim 20 wherein said platelet plasma and or said thrombin are autologous.

23. The method according to claim 20 wherein said platelet gel further comprises one or more bioactive agents selected from the group consisting of small molecules, peptides, proteins, hormones, DNA or RNA fragments, cells, genetically engineered cells, genes, cell growth promoting compositions and matrix metalloproteinase inhibitors.
